(19) United States
(12) Patent Application Publication
(10) Pub. No.: US 2004/0106214 A1
(43) Pub. Date: Jun. 3, 2004

(54) Dry analytical element

(76) Inventors: Yoshikazu Amano, Saitama (JP); Kazuya Kawasaki, Saitama (JP); Kentaro Nakamura, Saitama (JP)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/720,373

(22) Filed: Nov. 24, 2003

Related U.S. Application Data
(63) Continuation of application No. 09/858,392, filed on May 16, 2001, now abandoned.

(30) Foreign Application Priority Data
May 16, 2000 (JP) 2000/142729

(51) Int. Cl. G01N 33/558

(52) U.S. Cl. 436/514

(57) ABSTRACT

A dry analytical element for calorimetric or turbidity analysis comprising a water-impermeable sheet that comprises an analyte storable area containing a necessary reagent for a designed analysis in dry state, wherein a specimen containing an analyte develops to the whole planar directions in substantially equal volume when it is supplied on the sheet.

### FIELD OF THE INVENTION

This invention is directed to a dry analytical element that enables prompt and facile analysis of plural analytes using a small quantity of specimen in such a field as clinical diagnosis.

### BACKGROUND OF THE INVENTION

Diagnosis of human illness by analyzing such a specimen as blood or urine has been prosecuted for a long period of time. To analyze an analyte(s) in the specimen, there are two processes, i.e. a wet process and a dry process.

In wet process, a specimen including an analyte(s) and a necessary reagent(s) is mixed in liquid in a vessel to produce some reaction, and then generated change is determined.

Problems in the wet process are requirement for a larger quantity of specimen and lack in facility and promptness. That is, about 0.1 ml˜0.5 ml of specimen is required for each analysis, so a large quantity of specimen is necessary for plural analysis. Consequently, when blood is required as the specimen of analysis, a large burden is imposed on a test subject. Further, the wet process is troublesome and takes time since different reagents are added to each vessel, respectively, containing the specimen and a necessary analyzer gets bigger in the nature of things.

In the dry process, a dry analytical element is used. The element contains some reagents in dry state that are necessary to detect a prospective analyte in the specimen. Usually only the specimen is supplied onto the dry analytical element during an analysis. The analysis with the dry analytical element requires a smaller quantity of specimen, and is prosecuted with facility and promptness.

But, sometimes it is hard to detect a decrease of optical density in colorimetric analysis, or turbidity in agglutination reaction or coagulation reaction using a dry analytical element.

One purpose of this invention is to provide an analytical element that enables facile and prompt analysis with a small quantity of specimen. The other purpose of the invention is to provide an analytical element that enables to analyze such reactions as chemical reactions, enzyme reactions, immune reactions, agglutination reactions or coagulation reactions.

A conventional dry analytical element is characterized in large part by the existence of a developing layer. A specimen supplied onto the developing layer penetrates to the whole lateral directions in substantially equal volumes. Then the ingredients in the specimen are supplied to a layer provided adjacent to the developing layer in an approximately constant quantity per unit area of the layer to result in a reliable and reproducible analytical result. Thus, the conventional dry analytical element was accomplished by the invention of the developing layer.

However, the present inventors found that by using the conventional dry analytical element with the developing layer, it was rather impossible to determine a part of the optical density of a dye formed in the developing layer in a colorimetric analysis of low optical density, or also to determine turbidity measured by degree of light scattering. These problems are due to the fact that when the dry analytical element is irradiated through its transparent support, the developing layer functions as a reflecting plate that reflects light irregularly. Usually, the support of the dry analytical element comprises a flat sheet of transparent and impermeable to water, and is about 0.2 mm in thickness.

### SUMMARY OF THE INVENTION

The object of the present invention is to provide a dry analytical element that enables a quantitative analysis with promptness and facility using a small quantity of specimen even for a calorimetric analysis, or a turbidity measurement caused by agglutination or coagulation reaction.

The present inventors have discovered that a specimen solution supplied on a water-impermeable sheet comprising some concavities or grooves in it, or comprising a group of projections on the surface of it, develops to the whole planer directions in substantially equal volumes, and that the aforementioned object can be achieved using the sheet. Here, “develops to the whole planer directions in substantially equal volumes” is a functional term meaning that any measurement error will not occur caused by differences in volume developed in different directions.

### BRIEF DESCRIPTION OF THE DRAWINGS

FIG. 1 is a perspective view showing an embodiment of the dry analytical element in accordance with the present invention, which comprises concavities in a sheet.

FIG. 2 is a perspective view showing the development of a specimen solution supplied onto the dry analytical element.

FIG. 3 is a perspective view showing an embodiment of the dry analytical element in accordance with the present invention, which comprises grooves in a sheet.

FIG. 4 is a perspective view showing an embodiment of the dry analytical element in accordance with the present invention, which comprises a group of projections on a sheet.

### DESCRIPTION OF THE PREFERED EMBODYMENT

As the water-impermeable sheet, material used as a support in a conventional dry analytical element can be utilized. For example, a sheet made of polyethylene terephthalate (PET), polycarbonate from bisphenol A, polystyrene or cellulose esters (e.g. cellulose diacetate, cellulose triacetate, cellulose acetate propionate etc.) is preferable. It is about 50 μm˜1 mm, preferably about 60 μm˜1 mm and more preferably about 80 μm˜500 μm in thickness.

On the water-impermeable sheet, an analyte storable area is formed. It stores a supplied liquid specimen containing an analyte(s). In the areas, a necessary reagent(s) for designed analysis is provided in dry state. The analyte in the liquid specimen chemically reacts with the reagent to result in some kind of detectable change.

The analyte storable area is composed of concavities or grooves formed in the water-impermeable sheet, or a group of projections formed on it. The analyte storable area can store about 1 μl˜100 μl, preferably about 3 μl˜50μl, of the liquid specimen.

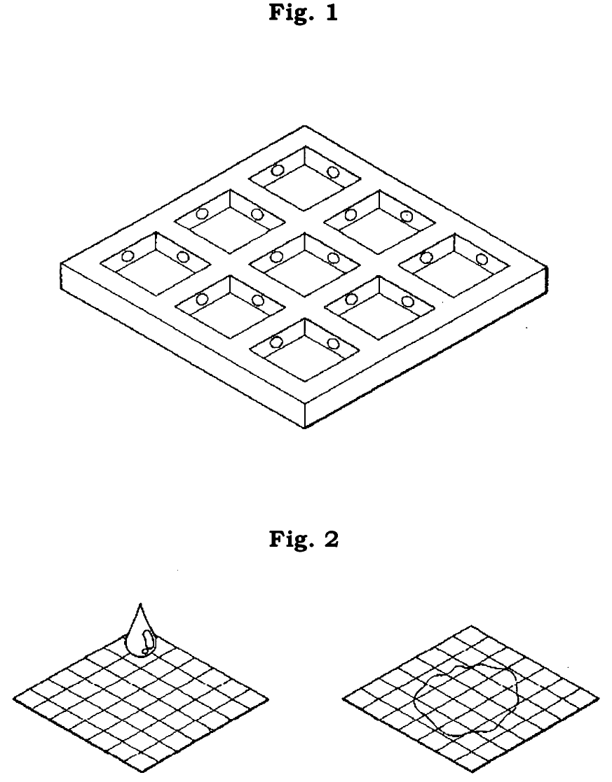

The concavity may have any arbitrary shape. As examples of planar shapes, a circle, a quadrangle or a hexagon may be mentioned. The bottom of the concavity is generally planar, but it may be concave or convex. The sidewall of it is usually vertical, but may be inclined. A through hole may be provided in the sidewall to permit the liquid specimen to move into a neighboring concavity. The diameter of the through hole is about 5 μm˜500 μm. The size of each concavity is about 50 μm˜7.5 mm, usually about 0.2 mm˜5.0 mm, in diameter of the circle or in side of the quadrangle. The depth of each concavity is about 10 m˜5.0 mm, usually about 30 μm˜2.0 mm at its center. The total number of the concavity in a sheet may be designed arbitrarily, and may be about 1˜500, preferably about 10˜200, more preferably about 10˜100. FIG. 1 shows a part of the sheet having concavities in it. FIG. 2 shows development of a liquid specimen supplied onto the sheet having concavities.

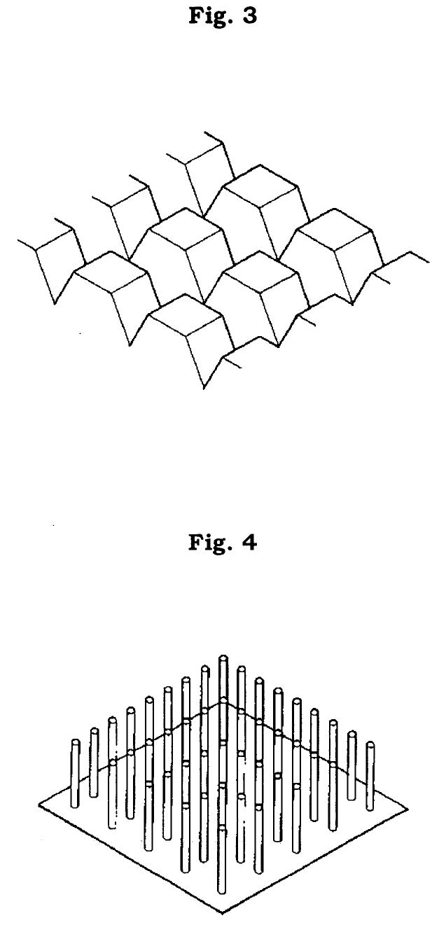

The groove also may have any arbitrary form, but preferably it is composed of a group of straight lines carved in parallel in the sheet, provided no particular object. These straight lines may be formed in more than one direction. In this case, a lattice of grooves may be formed in the sheet. Each verge of the groove may reach the border of the sheet or may not. The section of the groove also may be in any arbitrary shape. Horseshoe, U-shape, V-shape etc. may be mentioned as examples. A through hole may be formed in the sidewall of the groove permitting the liquid specimen to move into a neighboring groove. The diameter of the through hole is about 5 μm˜500 μm. Width of each groove at its top is about 50 μm˜7.5 mm, preferably about 200 μm˜5.0 mm. Depth of it is about 10 μm˜5.0 mm, preferably about 30 μm˜2.0 mm. Number of the grooves in the sheet may be arbitrarily designed, and is usually about 1˜500, preferably about 10˜100, and more preferably about 10˜50. FIG. 3 exemplifies the sheet with grooves in it.

The shape of each projection is not restricted if the group of projections can keep a designed quantity of liquid sample in the space. Each projection is about 10 μm˜1 mm, preferably about 50 μm˜500 μm diameter and 10 μm˜5.0 mm, preferably 50 μm˜2.0 μm in length (or in height). Number of the projections is about 10˜1000, preferably 25˜100. Planar dimension of the group of projections is about 1 mm²˜400 mm², preferably about 25 mm²˜200 mm². Total number of the group of projections formed on the sheet is about 1˜1000, preferably 25˜100 . FIG. 4 exemplifies the sheet with a group of projections on it.

Surface of the concavities or the grooves is preferably hydrophilic, considering faster spreading of liquid specimen supplied on it. A hydrophilic surface is obtained, for examples, by glow discharge treatment, treatment with surfactant or a solution of protein. All the surface of water impermeable sheet except for the analyte storable area may be hydrophilic, hydrophobic or water repellent.

There is no particular restriction of analytes to be analyzed using the dry analytical element prepared in accordance with the present invention. Enzymes, lipids, inorganic ions, metabolic products, proteins, which are usually analyzed in clinical diagnosis, are objects of analysis using the dry analytical element. Further, ingredients from living organism (globulins, immune antigens, immune antibodies etc. as examples), drugs, hormones, tumor markers, DNA or RNA are also objects of analysis using the dry analytical element, if the analytical method for each of them has been established.

The dry analytical element in accordance with this invention contains the all reagents necessary for a designed analysis on the analyte storable area. Here, “the all reagents necessary for a designed analysis” means critical reagents for a designed analysis, and other reagents may be added if necessary. The reagent(s) may be the same as that used in the known dry analytical elements. They are provided on the analyte storable area by coating, spraying or dropping the solution containing the necessary reagent(s). The solution may further contain some hydrophilic. polymer. Then the solution supplied onto the analyte storable area is dried with heating, reducing pressure or freezing, depending on the thermal stability of the reagent.

The quantity of the specimen supplied onto each sheet comprising the analyte storable area is about 1 μl˜100 μl, preferably about 3 μl˜50 μl. The sheet is then incubated under the known condition for conventional dry analytical element.

Photometry is prosecuted after the end of the reaction between the analyte and the reagent. Light may be exposed perpendicularly or obliquely onto the front surface of the sheet or from backside through the sheet. Either reflected or transmitted light may be measured, using a prism or a miller if necessary, in the photometry.

Thus, the dry analytical element in accordance with the present invention reveals advantage of the conventional wet analytical system in conjunction with the conventional dry analytical system. That is, it makes prompt and facile analysis possible with rather a little volume of specimen, even for calorimetric or turbidity analysis.

The present invention will be more clearly understood with reference to the following examples.

### EXAMPLES

### Example 1

In a colorless, transparent PET sheet (180 μm thick) square concavities of 1.0 mm in side and 0.1 mm in depth were formed. Distance between neighboring concavities was 0.4 mm. On the sheet comprising concavities, a reagent solution containing carboxymethyl starch, latex sensitized with anti-HCG antibody and cattle serum albumin was supplied and dried to form the sheet with analyte storable areas comprising a reagent layer.

The dried quantity of each reagent on the sheet was as follows.

carboxymethyl starch 5.8 g/m²

latex sensitized with anti-HCG antibody 1.6 g/m²

cattle serum albumin 1.6 g/m²

The sheet was cut into chips of 12×13 mm. Then each chip was mounted in the slide holder described in JP 1982-063452-A to form a dry analytical element according to the present invention.

Human Chorionic Gonadotropin (HCG manufactured by SIGMA) was diluted with a buffer solution of 100 mM phosphoric acid(pH7.4) to prepare four solutions of HCG. Each of the four solutions contains 0.5, 25, 100 or 500IU/ml HCG, respectively.

Then 10 μl of each solution was spotted onto four dry analytical elements mentioned above, respectively. Each dry analytical element was incubated at 37° C. for 5 minutes. After the incubation, light of 650 nm in wavelength was exposed through the sheet and reflective optical density was measured. During the measurement process, a black plate was put on the sheet to absorb light passed through the reagent layer.

Measured values were shown in Table 1 below. It is obvious that a quantitative determination of HCG is possible using the dry analytical element according to this invention.

TABLE 1

| Density of HCG [IU/ml] | Optical Density (at 650 nm) |
|---|---|
| 0 | 1.380 |
| 5 | 1.404 |
| 25 | 1.454 |
| 100 | 1.491 |
| 500 | 1.534 |

### Comparative Example 1

On the colorless, transparent PET sheet (180 μm thick) the reagent solution described in the example 1 was coated and dried to form a reagent layer. The dried quantity of each reagent on the sheet was as follows.

carboxymethyl starch 5.8 g/m²

latex sensitized with anti-HCG antibody 1.6 g/m²

cattle serum albumin 1.6 g/m²

The whole surface of the reagent layer was dampened with water of about 30 g/m². On the dampened surface of the reagent layer, a tricot knitted cloth formed by knitting 50 denier PET spun yarn with 36 gauge was laminated with a light pressure. Then the laminate on the sheet was dried.

The sheet was cut into chips of 12×13 mm. Then each chip was mounted in the slide holder described in JP 1982-063452-A to form a comparative dry analytical element.

Then HCG was determined according to the same process and using four solutions described in the example 1.

Measured values were shown in Table 2 below. It is obvious that a quantitative determination of HCG is impossible using the comparative dry analytical element.

TABLE 2

| Density of HCG [IU/ml] | Optical Density (at 650 nm) |
|---|---|
| 1 | 0.310 |
| 6 | 0.313 |
| 26 | 0.308 |
| 101 | 0.318 |
| 500 | 0.308 |

1. A dry analytical element comprising a water-impermeable sheet that comprises an analyte storable area containing a necessary reagent for a designed analysis in dry state, wherein a specimen liquid containing an analyte develops in
whole planar directions in substantially equal volume when it is supplied on the sheet.

2. The dry analytical element as claimed in claim 1, wherein the analyte storable area comprises a group of concavities.

3. The dry analytical element as claimed in claim 1, wherein the analyte storable area comprises a group of grooves.

4. The dry analytical element as claimed in claim 1, wherein the analyte storable area comprises a group of projections.

5. The dry analytical element as claimed in claim 1, wherein the specimen liquid is plasma or serum.
